(19) United States
(12) Patent Application Publication
(10) Pub. No.: US 2023/0282307 A1
(43) Pub. Date: Sep. 7, 2023

(54) METHOD FOR DETECTING UNIPARENTAL DISOMY BASED UPON NGS-TRIO, AND USE THEREOF

(71) Applicants: Guangzhou Kingmed Center for Clinical Laboratory, Guangzhou, Guangdong (CN); Guangzhou Kingmed Diagnostics Group Co., Ltd., Guangzhou, Guangdong (CN)

(72) Inventors: Jingxing LIU, Guangzhou, Guangdong (CN); Shihui YU, Guangzhou, Guangdong (CN); Changshun YU, Guangzhou, Guangdong (CN); Lina XIANG, Guangzhou, Guangdong (CN); Baixue CHEN, Guangzhou, Guangdong (CN)

(21) Appl. No.: 18/019,858

(22) PCT Filed: Aug. 4, 2020
(86) PCT No.: PCT/CN2020/106716

§ 371 Date: Feb. 6, 2023

(51) Int. Cl. G16B 30/00; G16B 40/20; G16H 50/20

(52) U.S. Cl. CPC G16B 30/00; G16B 40/20; G16H 50/20

(57) ABSTRACT

A method for detecting a uniparental disomy based upon NGS-trio and a use thereof, which belongs to the technical field of bioinformatics analysis is provided. The method can directly deduce the genetic origin of chromosome for proband through obtaining NGS-trio sequencing data, analyzing and judging, so as to directly judge whether UPD occurs in the sample (rather than indirectly deduce whether UPD occurs in the sample through LOH), thereby improving positive diagnosis rate without increasing any cost. Further, the method also can assist in the judgment of loss of heterozygosity of large fragments, and its resolution can reach 1 Mbp according to the density of mutation sites, showing excellent detection performance.

### FIELD OF TECHNOLOGY

The present disclosure relates to the technical field of bioinformatics analysis, particularly, it relates to a method for detecting a uniparental disomy based upon NGS-trio and a use thereof.

### BACKGROUND

Genomic imprinting, also known as genetic imprinting, is a genetic process where one gene or genomic region is marked in accordance to its parent of origin through a biochemical approach. The gene is named as an imprinted gene whose expression depends on the origin (paternal line and maternal line) of chromosome which the gene is located in and depends on whether the gene is silenced (the silencing mechanism is mostly methylation) on the chromosome from which it is originated. Some imprinted genes are only expressed in maternal chromosomes, while some others are expressed in paternal chromosomes.

In a normal diplont, one chromosome of each homologous pair comes from the father and one comes from the mother. UniParental Disomy (UPD for short) refers to a situation where a pair of homologous chromosomes (or some regions on the chromosome) comes from only one parent. If such regions include imprinted genes, they may result in disordered expression of the genes. At present, the methylation level detection method is to detect whether the methylation levels of the same regions on a pair of homologous chromosomes are the same.

In most cases, UPD is caused by two non-disjunction homologous chromosomes during meiosis, therefore producing gamete with abnormal copy number of chromosomes. Compared with the normal gamete having one copy, the abnormal gamete has two copies or no copy, thus producing zygote with abnormal copy number (trisomy or monosomy). Finally, euploid is regained through trisomy rescue, that is, by randomly losing one chromosome, as shown in FIG. 1;

alternatively, euploid is regained through monosomy rescue, that is, by copying one monosomy, as shown in FIG. 2. The trisomy rescue may have one in three probability of producing UPDs, but monosomy rescue certainly produces UPDs.

The UPDs produced by monosomy rescue can be indirectly detected and deduced by LOH (loss of heterozygosity) detection, because of the homozygosity of the entire chromosome. For the UPDs produced by trisomy rescue, although local LOH may occasionally occur due to recombination during meiosis, there are many reasons to produce local LOH (such as consanguineous marriage), and therefore it is not 100% clear that UPDs are produced.

However, in the common prior art, the methylation method for detecting UPDs can only deal with small regions on a part of chromosomes, and different experiments are required to be designed for different regions, which results in low efficiency and is not suitable for a genome-wide screening; As for the SNP chip-based method, it has the disadvantage of high cost, and its targeted probes comprise polymorphism sites, so pathogenic micro-mutations (point mutations, small insertions/deletions) cannot be detected at the same time.

Currently, Whole exome sequencing (WES) is the most common method for detecting gene defeat diseases, which can detect pathogenic point mutations, micro-insertion loss, copy number variations etc., and therefore it is a preferred option for patients suffering from such diseases. However, as disclosed in CN 110211630A, UPDs could only be deduced indirectly by LOH, based on sequencing data of a single sample.

### SUMMARY

Based on this, it is necessary to address the above-mentioned problem and provide a method for detecting a uniparental disomy based upon NGS-trio, which can directly deduce the genetic origin of chromosome for proband, so as to directly determine whether UPDs occur (rather than indirectly deduce whether UPDs occur by LOH), thereby improving positive diagnosis rate without increasing any cost.

A method for detecting a uniparental disomy based upon NGS-trio comprises the steps as follows:

obtaining data: obtaining NGS sequencing data of trio-samples in a same sample group;

screening for mutation sites: selecting mutation sites which are in conformity with pre-determined conditions in each trio-sample, respectively and defining such mutation sites as qualified mutation sites of corresponding trio-samples, and defining un-selected mutation sites as unqualified mutation sites;

merging mutation site data: merging the unqualified mutation sites from all the trio-samples in the same sample group, obtaining and gathering a chromosome coordinate of each unqualified mutation site, removing mutation sites which have identical chromosome coordinate to those of the unqualified mutation sites from the qualified mutation sites in each trio-sample; and based on the remaining qualified mutation sites of the samples in the sample group, defining genotypes of non-mutation sites as genotypes of homozygous sites, which are consistent with genotypes of the reference sequence;

classifying inheritance pattern: classifying inheritance patterns for the trio-sample combinations at each mutation site, wherein the mutation sites can be classified into loci in conformity with biparental inheritance, loci in conformity with uniparental inheritance only, and loci in inconformity with heredity law;

judging genetic relationship: if the number of the loci in inconformity with heredity law is smaller than a pre-set value, a follow-up analysis is performed; if the number of the loci in inconformity with heredity law is larger than the pre-set value, the sample is judged to be unqualified;

judging uniparental fragment: if a coverage of consecutive loci which are only in conformity with uniparental paternal inheritance exceed a pre-set value, the fragment is judged to be a uniparental paternal fragment; if the coverage of consecutive loci which are only in conformity with uniparental maternal inheritance exceed a pre-set value, the fragment is judged to be a uniparental maternal fragment;

judging UPD: analyzing depth-of-coverage of sequencing data of the judged uniparental fragment, wherein if the judged uniparental fragment contains a single copy, it can be judged that fragment deletion occurs in the uniparental fragment; if the judged uniparental fragment does not contain a single copy, the uniparental fragment is judged as a UPD fragment;

screening pathogenic UPD: determining whether the UPD fragment covers imprinted gene or corresponding band, wherein if the UPD fragment does not cover the imprinted gene or corresponding band, the UPD fragment is judged to be benign UPD, if the UPD fragment covers the imprinted gene or corresponding band, the UPD fragment is judged to be pathogenic UPD.

Since the cost of sequencing has decreased, an increasing number of WES sequencing methods detect samples from both proband and their parents. Based on such trio database system of families, the above method can directly deduce the genetic origin of chromosome from the proband, so as to directly determine whether the chromosome is UPD and thus improve positive diagnosis rate without increasing any cost.

It can be understood that, the above-described NGS data can be either whole-exome sequencing data or whole-genome sequencing data.

In one example, in the step of screening for mutation sites, the mutation sites are obtained as follows:

1) screening for high-quality mutation sites from NGS sequencing data;

2) removing Y chromosome mutation sites from the above mutation sites;

3) screening for single nucleotide substitutions from the mutation sites obtained in the step of removing Y chromosome mutation sites;

4) excluding uncertain false positive single nucleotide substitutions according to Hardy-Weinberg equilibrium;

5) removing heterozygous sites which have a mutation frequency of more than 70% and homozygous sites which have a mutation frequency of less than 85%;

6) classifying a genotype of the mutation for each site, and removing sites with more than 2 genotypes;

7) determining remaining sites, which meet the predetermined conditions.

In mutation analysis, as human have diploid, there are at most two genotypes at one site. If there are more than two genotypes at one site, it is generally caused by sequencing errors. For example, a genotype of chr1:69849G>A (Het, heterozygous) is chr1:69849[A/G], and a genotype of chr1:69849G>A (Hom, homozygous) is chr1:69849[A/A]. When there are chr1:69849G>A (Het) and chr1:69849G>T (Het) presented together, the genotype is chr1:69849[A/G/T], that is, such site has more than 2 genotypes, and therefore it should be removed.

It can be understood that, said mutation sites in conformity with predetermined conditions should meet all of the above screening conditions and fail to meet all of the above removing conditions at the same time.

It can be understood that, according to Hardy-Weinberg equilibrium, when a population is infinite, randomly mating, with non-mutation, non-selection and non-genetic drift, the frequency of genotype and frequency of gene in a locus in the population will remain unchanged from generation to generation and reach genetic equilibrium. Therefore, a false positive locus can be excluded by chi-square test. For example, the frequency of AA-AB-BB is regular. For example, in a local population database of 10000 people, if the frequency of A allele is 0.4 and the frequency of B allele is 0.6, the theoretical number of people with AA genotype, BB genotype and AB genotype is 1600, 3600, and 4800, respectively. Chi-square test is performed based on the actual number and theoretical number of people with these genotypes in the population database, and the loci where the actual number is deviated far away from the theoretical number (i.e., highly suspected false positive loci) will be excluded.

A large number of loci with poor quality are comprised in the results of conventional NGS sequencing, which will greatly interfere with the subsequent step of judging UPD in the above method. If all loci are used, the detection effect is poor. Therefore, the accuracy of the analysis result can be improved by selecting the mutation loci according to the above method.

In one example, in the step of screening for mutation sites, the high-quality mutation sites are those passed through a quality control of GATK-VQSR, and having a total coverage range of more than 20× and a mutation frequency of greater than 25%.

In one example, in the step of obtaining data, the trio samples in the same group comprise a paternal sample, a maternal sample and a proband sample.

In the step of merging mutation site data, the mutation sites, which have identical coordinate, are arranged in an order of proband, father, mother.

If the detection is performed in accordance with the method of the present disclosure, a proband sample, a paternal sample, and a maternal sample must be included, none of them can be dispensed.

In one example, in the step of classifying inheritance pattern, the loci in conformity with biparental inheritance can be classified into:

Type 1: loci only in conformity with biparental inheritance;

Type 0: loci in conformity with both biparental inheritance and uniparental inheritance; the loci in conformity with uniparental inheritance only can be classified into:

Type 3F: loci only produced by paternal monosomy rescue;

Type 2F: loci produced by either paternal monosomy rescue or paternal trisomy rescue;

Type 3M: loci only produced by maternal monosomy rescue;

Type 2M: loci produced by either maternal monosomy rescue or maternal trisomy rescue; the loci in inconformity with heredity law can be classified into:

Type −1: loci from either of parent in inconformity with heredity law;

Type −2: loci from both parents in inconformity with heredity law.

It can be understood that, “the loci in conformity with biparental inheritance” refers to the loci where the origin of two alleles from the proband can be found in both parents, and includes the loci only in conformity with biparental inheritance (i.e., Type 1, such as Aa-AA-aa), as well as the loci in conformity with both biparental inheritance and uniparental inheritance (i.e., Type 0).

In one example, in the step of judging uniparental fragment, if there are more than 8 Type 2F loci or Type 3F loci with a coverage of more than 1 Mbp, the fragment is judged to be a uniparental paternal fragment; if there are more than 8 Type 2M loci or Type 3M loci with a coverage of more than 1 Mbp, the fragment is judged to be a uniparental maternal fragment.

It can be understood that, the above consecutive loci are not separated by Type 1 loci. For example, more than eight consecutive Type 2F loci or Type 3F loci are not separated by Type 1 loci; alternatively, more than eight consecutive Type 2M loci or Type 3M loci are not separated by Type 1 loci.

In one example, in the step of judging UPD, the data of the judged uniparental fragment is compared with the analysis results of copy number of whole exome sequencing, and if the analysis result of copy number indicates that the judged uniparental fragment contains a single copy, it can be judged that fragment deletion occurs in the uniparental fragment; if not, the uniparental fragment is judged to be a UPD fragment.

The present disclosure further discloses a use of the above-mentioned method of detecting a uniparental disomy based upon NGS-trio in developing or manufacturing a device for screening UPD.

The present disclosure further discloses a device for screening a uniparental disomy based upon NGS-trio, and the device comprises a module of obtaining data, a module of analyzing data, and a module of judging UPD; wherein

the module of obtaining data is used to obtain NGS sequencing data of trio samples in a same group;

the module of analyzing data is used to analyze the above obtained data and classify mutation sites into loci in conformity with biparental inheritance, loci in conformity with uniparental inheritance only, and loci in inconformity with heredity law;

the module of judging UPD is used to perform UPD judgement on the above mutation sites according to a predetermined rule, to obtain a judgement result;

the module of analyzing data is conducted in following steps:

screening for mutation sites: selecting mutation sites which are in conformity with pre-determined conditions in each trio-sample, respectively and defining such mutation sites as qualified mutation sites of corresponding trio-samples, and defining un-selected mutation sites as unqualified mutation sites;

merging mutation site data: merging all the unqualified mutation sites from the trio-samples in the same sample group, obtaining and gathering chromosome coordinates of each unqualified mutation site, removing mutation sites which have identical chromosome coordinates to those of the unqualified mutation sites from the qualified mutation sites in each trio-sample; and based on the remaining qualified mutation sites in this group of the samples, defining a genotype of the non-mutation sites as a homozygous locus, which is consistent with the reference sequence;

classifying inheritance pattern: classifying inheritance patterns for trio-sample combinations at each mutation site, wherein the mutation sites can be classified into loci in conformity with biparental inheritance, loci in conformity with uniparental inheritance only, and loci in inconformity with heredity law;

the module of judging UPD is conducted in following steps:

judging genetic relationship: if the number of the loci in inconformity with heredity law is smaller than a pre-set value, a follow-up analysis is performed; if the number of the loci in inconformity with heredity law is larger than the pre-set value, the sample is judged to be unqualified;

judging uniparental fragment: if a coverage of consecutive loci which are only in conformity with uniparental paternal inheritance exceed a pre-set value, the fragment is judged to be a paternal fragment; if the coverage of consecutive loci which are only in conformity with uniparental maternal inheritance exceed a pre-set value, the fragment is judged to be a maternal fragment;

judging UPD: analyzing depth-of-coverage of the sequencing data of the judged uniparental fragment, wherein if the judged uniparental fragment contains a single copy, it can be judged that fragment deletion occurs in the uniparental fragment; if the judged uniparental fragment does not contain a single copy, otherwise, the uniparental fragment is judged as a UPD fragment;

screening pathogenic UPD: determining whether the UPD fragment covers imprinted gene or corresponding band, wherein if the UPD fragment does not cover the imprinted gene or corresponding band, the UPD fragment is judged to be benign UPD, if the UPD fragment coverages imprinted gene or corresponding band, the UPD fragment is judged to be pathogenic UPD.

In one example, in the step of screening for mutation sites, the mutation sites are obtained as follows:

1) screening for high-quality mutation sites from NGS sequencing data;

2) removing Y chromosome mutation sites from the above mutation sites;

3) screening for single nucleotide substitutions from the mutation sites obtained in the step of removing Y chromosome mutation sites;

4) excluding uncertain false positive single nucleotide substitutions according to Hardy-Weinberg equilibrium;

5) removing heterozygous sites which have a mutation frequency of more than 70% and homozygous sites which have a mutation frequency of less than 85%;

6) classifying a genotype of the mutation at each site, and removing sites with more than 2 genotypes;

7) determining remaining sites, which meet the predetermined condition.

In one embodiment, in the step of screening for mutation sites,

the high-quality mutation sites are those passed through a quality control of GATK-VQSR, and having a total coverage range of more than 20X and a mutation frequency of greater than 25%.

In one example, in the module of obtaining data, the trio samples in the same group comprise a paternal sample, a maternal sample and a proband sample.

In the step of merging mutation site data, the mutation sites, which have identical coordinate, are arranged in an order of proband, father, mother.

In one example, in the step of classifying inheritance pattern, the loci in conformity with biparental inheritance can be classified into:

Type 1: loci only in conformity with biparental inheritance;

Type 0: loci in conformity with both biparental inheritance and uniparental inheritance; the loci only in conformity with uniparental inheritance can be classified into:

Type 3F: loci only produced by paternal monosomy rescue;

Type 2F: loci produced by either paternal monosomy rescue or paternal trisomy rescue;

Type 3M: loci only produced by maternal monosomy rescue;

Type 2M: loci produced by either maternal monosomy rescue or maternal trisomy rescue; the loci in inconformity with heredity law, can be classifying into:

Type −1: loci from either of parent in inconformity with heredity law

Type −2: loci from both parents in inconformity with heredity law.

It can be understood that, “the loci in conformity with biparental inheritance” refers to the loci where the origin of two alleles from the proband can be found in both parents, and includes the loci only in conformity with biparental inheritance (i.e., Type 1, such as Aa-AA-aa), as well as the loci in conformity with both biparental inheritance and uniparental inheritance (i.e., Type 0).

In one example, in the step of judging uniparental fragment, if there are more than 8 Type 2F loci or Type 3F loci with a coverage of more than 1 Mbp, the fragment is judged to be uniparental paternal fragment; if there are more than 8 Type 2M loci or Type 3M loci with a coverage of more than 1 Mbp, the fragment is judged to be uniparental maternal fragment.

In one example, in the step of judging UPD, the data of the judged uniparental fragment is compared with the analysis results of copy number of whole exome sequencing, and if the analysis result of copy number indicates that the judged uniparental fragment contains a single copy, it can be judged that fragment deletion occurs in the judged uniparental fragment; if not, the uniparental fragment is judged to be a UPD fragment.

The present disclosure further discloses a storage medium, comprising a stored program which achieves functions of the above-mentioned modules.

The present disclosure further discloses a processor, which is used for running a program that realizes the functions of the above-mentioned modules.

Compared with the prior art, the present disclosure has the benefits as follows:

The method for detecting uniparental disomy based upon NGS-trio of the present disclosure is based on trio data from whole exome/whole genome sequencing (NGS-trio), and can judge whether UPD occurs and whether UPD occurs in high-risk imprinted regions while examining common pathogenic mutations, without additional experiments and labor cost.

In addition, this method also can be used to assist in the judgment of loss of heterozygosity (LOH) of large fragments, and its resolution can reach 1Mbp according to the density of mutation sites, showing excellent detection performance.

### BRIEF DESCRIPTION OF FIGURES

FIG. 1 depicts a schematic diagram of trisomy rescue in the BACKGROUND;

FIG. 2 depicts a schematic diagram of monosomy rescue in the BACKGROUND;

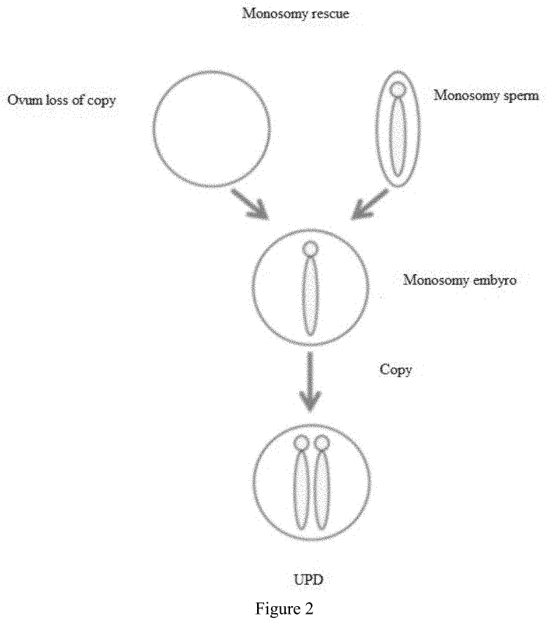

FIG. 3 depicts a flow chart of a method for detecting a uniparental disomy based upon NGS-tri according to Example 1;

FIG. 4 depicts a schematic diagram of modules of a screening device in Example 2;

FIG. 5 depicts a schematic diagram of normal samples in Example 3.

FIG. 6 depicts a schematic diagram of the analysis of a trio sample group, NP21S0557-NP21S0558-NP21S0549, in Example 4.

FIG. 7 depicts an enlarged schematic diagram of a circled portion of FIG. 4.

FIG. 8 depicts a schematic diagram of the analysis of a trio sample group, NP19E0911-NP19E0910-NP19E0912, in Example 4.

FIG. 9 depicts an enlarged schematic diagram of a circled portion of FIG. 6.

FIG. 10 depicts a schematic diagram of the analysis of a trio sample group, NP20E957-NP20E956-NP20E958, in Example 4.

FIG. 11 depicts an enlarged schematic diagram of a circled portion of FIG. 8.

FIG. 12 depicts a schematic diagram of the analysis of a trio sample group, NP21F6166 -

NP21F6167 -NP21F6168, in Example 5.

FIG. 13 depicts an enlarged schematic diagram of a circled portion of FIG. 10.

FIG. 14 depicts a schematic diagram of the analysis of a trio sample group, NP19F0315 -NP19F0313 -NP19F0314, in Example 5.

FIG. 15 depicts an enlarged schematic diagram of a circled portion of FIG. 12.

FIG. 16 depicts a schematic diagram of the analysis of a trio sample group, NP21F3536 -NP21F3567 -NP21F3537, in Example 5.

FIG. 17 depicts an enlarged schematic diagram of a circled portion of FIG. 14.

FIG. 18 depicts a schematic diagram of the analysis of a trio sample group, NP19E1380 -NP19E1381 -NP19E1382, in Example 6; and

FIG. 19 depicts an enlarged schematic diagram of a circled portion of FIG. 16.

FIG. 20 depicts a schematic diagram of the analysis of a trio sample group, NP19E0056 -NP9E0057 -NP9E0055, in Example 6; and

FIG. 21 depicts an enlarged schematic diagram of a circled portion of FIG. 18;

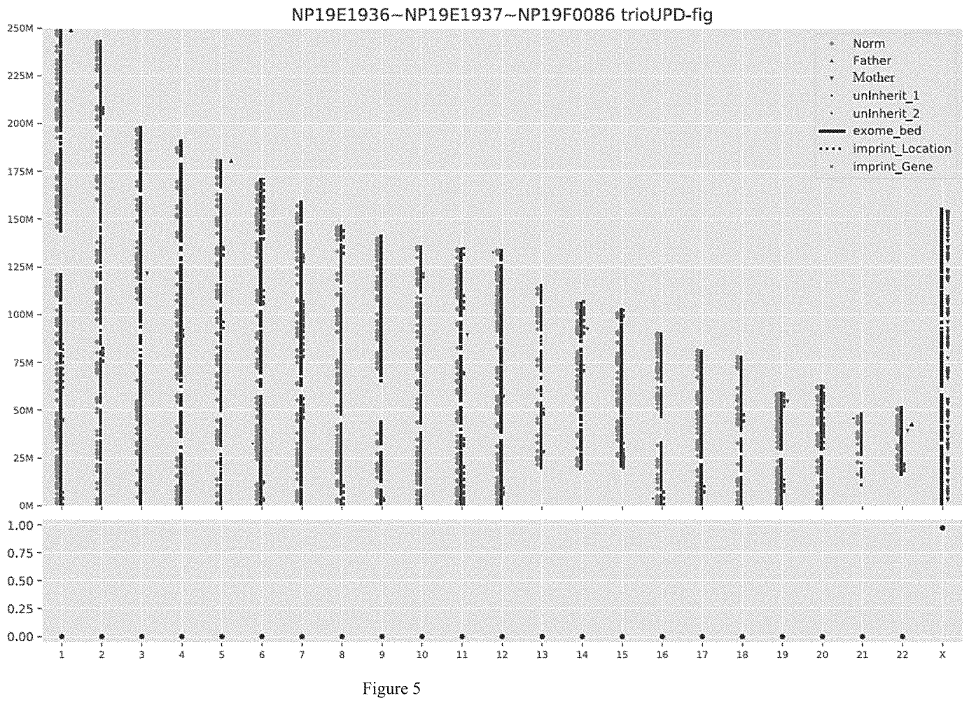

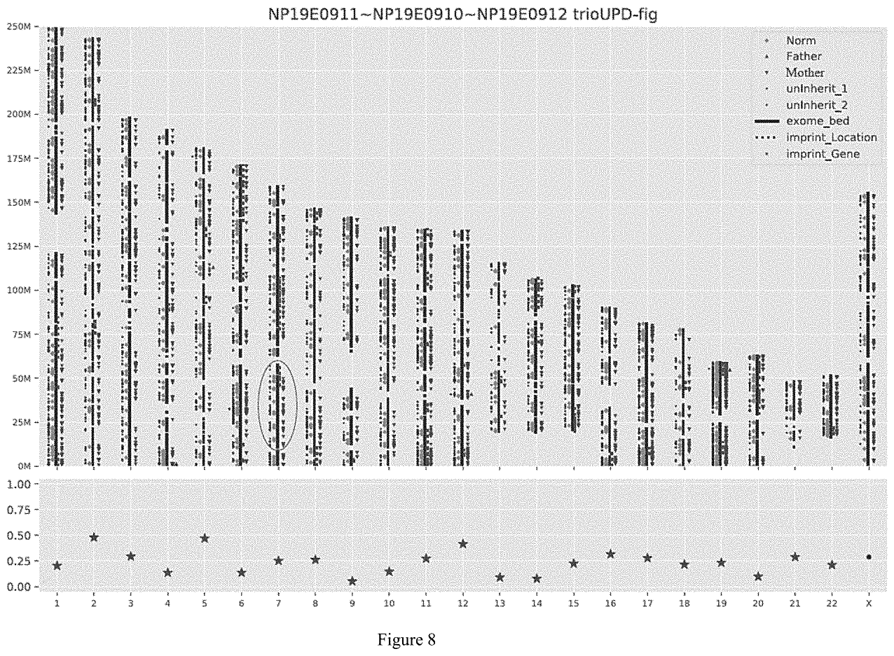

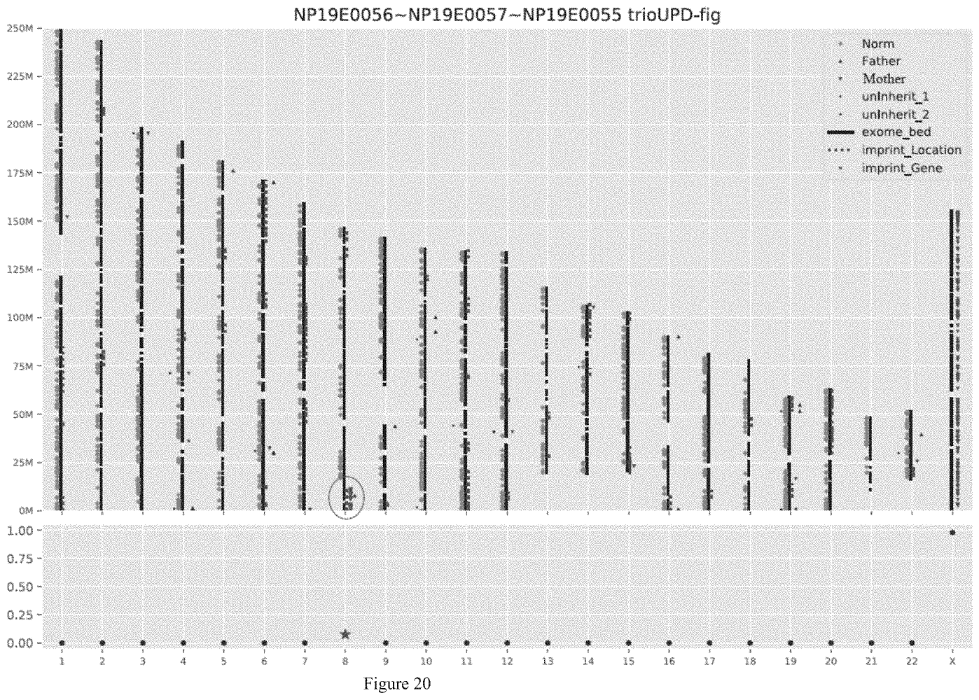

wherein, in FIGS. 5, 6, 6, 8, 10, 12, 14, 16, 18, 20, the abscissa indicates a serial number of each chromosome, the lower half of the figures shows proportions of lengths of contiguous homozygous sites to the entire chromosome, while the upper half of the figures shows the distribution of mutation sites on each chromosome; and

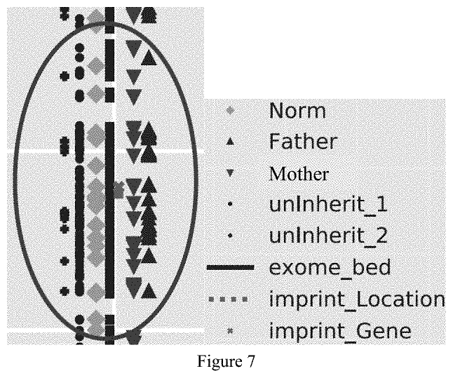

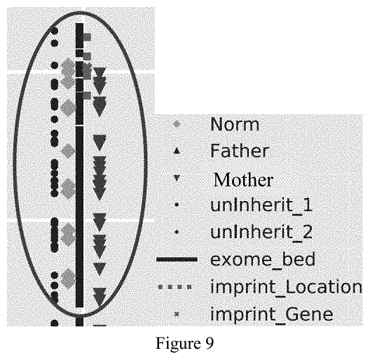

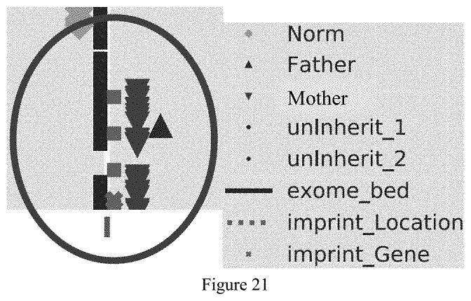

in the enlarged schematic diagrams of FIGS. 7, 9, 11, 13, 15, 17, 19 and 21, different types of loci at each chromosome are shown in an order from left to right of the schematic diagrams as follows: “cross unInherit2” refers to the Type −2 loci, “round dot unInherit_1” refers to the Type −1 loci, “diamond Norm” refers to the normal loci, “solid line exome_bed” refers to the full exome sequencing coverage, “imprint location” is the imprinted region, “imprint gene” is the imprinted gene coverage, “inverted triangle Mather” refers to the loci inherited from uniparental maternal inheritance (3M and 2M), and “equilateral triangle Father” refers to the loci inherited from uniparental paternal inheritance.

### DETAILED DESCRIPTION

For better understanding of the present disclosure, the present disclosure will be fully described below with reference to the relevant accompanying figures. The preferred embodiments are shown in the figures. However, the present disclosure can be implemented in many different forms and is not limited to the embodiments described herein. Rather, these embodiments are provided for the purpose of making the disclosed contents of the present disclosure more thorough and complete.

Unless otherwise defined, all technical and scientific terms used herein have the same meaning as those normally understood by one skilled in the art in the technical field belonging to the present disclosure. The terms used in the description of the present disclosure herein are only for the purpose of describing embodiments, and are not intended to limit the present disclosure. The term “and/or” used herein comprises anyone or all combinations of one or more corresponding items listed herein.

### Example 1

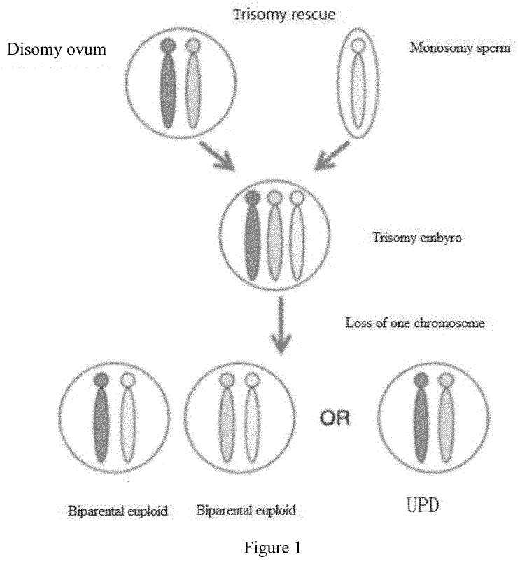

A flow chart of a method for detecting a uniparental disomy based upon NGS-trio was shown in FIG. 1, and the method comprises the steps as follows:

I. Obtaining Data

NGS sequencing data of trio samples in a same group was obtained. It can be understood that, such NGS sequencing data could be either whole exome sequencing data or whole genome sequencing data.

For the samples, a proband sample, a paternal sample and a maternal sample are all required.

II. Screening for Mutation Sites.

For a trio sample group, mutation sites which are in conformity with the predetermined conditions in each trio-sample were selected separately, and defined as qualified mutation sites of the corresponding samples, and the un-selected mutation sites were defined as unqualified mutation sites. Specifically, the screening step was performed according to the following process:

1. screening for high-quality mutation sites (the high-quality mutation sites are those passed through a quality control of GATK-VQSR, and having a total coverage range of more than 20X and a mutation frequency of greater than 25%) in whole exome sequencing.

2. removing Y chromosome mutation sites from the above mutation sites

3. screening for single nucleotide substitutions from the mutations obtained in the step of removing Y chromosome mutation sites;

4. excluding uncertain false positive single nucleotide substitutions in a frequency database for local population, according to Hardy-Weinberg equilibrium;

5. removing heterozygous sites which have a mutation frequency of more than 70% and homozygous sites which have a mutation frequency of less than 85%;

6. classifying a genotype of the mutation at each site, and removing sites with more than 2 genotypes, (as humans are diploid, there are at most 2 genotypes at one site; if there are more than 2 genotypes at one site, it is generally caused by sequencing errors). For example, a genotype of chr1:69849G>A (Het) is chr1:69849[A/G], and a genotype of chr1:69849G>A (Hom) is chr1:69849[A/A]. When there are chr1:69849G>A (Het) and chr1:69849G>T (Het) presented together, the genotype is chr1:69849[A/G/T], that is, such site has more than 2 genotypes, and therefore it should be removed.

7. The above qualified sites and unqualified sites in the above screening step were merged and recorded, respectively.

The qualified sites should “meet all of the above screening conditions” and “fail to meet all of the above removing conditions” at the same time.

III. Merging Mutation Site Data

1. The unqualified mutation sites of three trio-samples (the proband, the father and the mother samples) in the same group were merged, and then a chromosomal coordinate of each unqualified mutation site was obtained and gathered, mutation sites which have identical chromosome coordinate to those of the unqualified mutation sites were removed from the qualified mutation sites in each trio-sample, that is, as long as a site was unqualified in a sample, it should also be removed in the other two samples.

2. Then, based on the remaining qualified mutation sites of the samples in this group, the genotype of the non-mutation sites was defined as a genotype of the homozygous site, which was consistent with the genotype of the reference sequence. For example, if the genotype of the proband chr1 is chr1:69849[A/G], the genotype of the father chr1 is chr1:69849[A/A], and there is no mutation at mother chr1, the genotype of mother chr1 should be chr1:69849[G/G], because the reference sequence of chr1 at that site is G.

After the above processing, the whole-exome sequencing data generally yielded about 50,000 eligible trio-sample combinations of the mutation sites. And the trio-sample combinations of the mutation sites were arranged in the following order: proband-father-mother, e.g. Aa-AA-aa, i.e., Aa was for the proband, AA was for the father and aa was for the mother.

IV. Classifying Inheritance Pattern

Inheritance patterns for trio-sample combinations were classified at each mutation site, wherein the mutation sites could be classified into loci in conformity with biparental inheritance, loci in conformity with uniparental inheritance only, and loci in inconformity with heredity law. Specifically, the mutation sites could be classified as follows:

1. Loci in conformity with biparental inheritance, i.e., the loci where the origin of two alleles from the proband can be found in both parents, wherein the type of Aa-AA-aa must be in conformity with biparental inheritance, and such loci were labeled as Type 1 (loci which are only in conformity with biparental inheritance). Although other types, such as Aa-Aa-Aa and AA-AA-Aa etc., were also in conformity with biparental inheritance, they were also in conformity with uniparental inheritance, and could not be used as the basis for any judgment, and therefore were labeled as Type 0 (loci which were in conformity with both biparental inheritance and uniparental inheritance).

2. Loci in conformity with uniparental inheritance only, i.e., the loci where two alleles from the proband were only inherited from one of parents. As exemplified by the alleles which were only inherited from father, there are two types, that is, AA-AA-aa and AA-Aa-aa, wherein the type of AA-Aa-aa was only generated by monosomy rescue and was labeled as Type 3F, and the type of AA-AA-aa was generated by either monosomy rescue or trisomy rescue, and was labeled as Type 2F. Similarly, if the alleles were only inherited from mother, the corresponding types were labeled as Type 3M and Type 2M, respectively.

3. Loci in inconformity with heredity law: if there were several sporadic mutation sites, gene mutation might happen in the genetic process, or there were sequencing errors; if there are a broad range of mutation sites, the parents might be non-biological. There are two situations, i.e., the type of AA-aa-aa, which meant that both parents were non-biological and was labeled as Type −2, and the type of Aa-aa-aa, which meant that one of parents was non-biological and was labeled as Type

V. Judging Genetic Relationship

If the number of the loci in inconformity with heredity law was smaller than a pre-set value, a follow-up analysis was performed; if the number of the loci in inconformity with heredity law was larger than the pre-set value, the sample was judged to be unqualified.

Normally, a small number of Type −1 loci and Type −2 loci might be produced sporadically, due to gene mutation and sequencing errors, and the number of such loci were less than 100 in general. However, for non-biological case, even if only one of the parents were non-biological, there were thousands of Type -1 loci.

In conclusion, if there were more than 800 Type −1 loci and Type −2 loci, the parents could be considered as being non-biological, that is, in the Example 1, the pre-set value (threshold value) of the loci in inconformity with heredity law was set to be 800.

If the genetic relationship of the samples was non-biological, the follow-up analysis was stopped. If the samples fulfilled the requirement in the judgment of the genetic relationship, the following process was continued.

VI. Judging Uniparental Fragment

If a coverage of consecutive loci which were only in conformity with uniparental paternal inheritance exceeded a pre-set value, the fragment was judged to be a uniparental paternal fragment; if the coverage of consecutive loci which were only in conformity with uniparental maternal inheritance exceeded a pre-set value, the fragment was judged to be a uniparental maternal fragment.

Specifically, in the Example 1, uniparental paternal/maternal fragments were judged as follows: if there were more than 8 consecutive Type 2F loci or Type 3F loci (i.e., the 8 consecutive loci were not separated by Type 1 loci) with a coverage of more than 1 Mbp in a fragment, the fragment was judged to be a uniparental paternal fragment. Similarly, there were more than 8 consecutive Type 2M loci or Type 3M loci (i.e., the 8 consecutive loci were not separated by Type 1 loci) with a coverage of more than 1 Mbp in a fragment, the fragment was judged to be a uniparental maternal fragment.

VII. Judging UPD

The depth-of-coverage of sequencing data of the judged uniparental fragment was analyzed. If the judged uniparental fragment contained a single copy, it can be judged that fragment deletion occurred in the uniparental fragment; otherwise, the uniparental fragment was judged as a UPD fragment. Specifically, the process was conducted as follows.

The depth-of-coverage of sequencing data of the judged uniparental fragment was analyzed in combination with the analysis results of whole exome sequencing copy number variation (CNV),and the depth-of-coverage of the sequencing data of the above uniparental paternal/maternal fragment was compared with the depth-of-coverage of the sequencing data of other samples sequenced in the same batch. If the CNV analysis suggested that the fragment contained a single copy, it was judged that fragment deletion occurred in the fragment. If not, the fragment was judged as UPD. In particular, large deletions were usually lethal, therefore, if the deletion reached more than half of the whole chromosome or even the whole chromosome, and the sample is non-embryonic, fragment deletions could be excluded basically.

VIII. Screening Pathogenic UPD

It was determined whether the UPD fragment covered imprinted gene or corresponding band. If the UPD fragment did not cover imprinted gene or corresponding band, the UPD fragment was judged to be benign UP. If the UPD fragment covered imprinted gene or corresponding band, the UPD fragment was judged to be pathogenic.

### Example 2

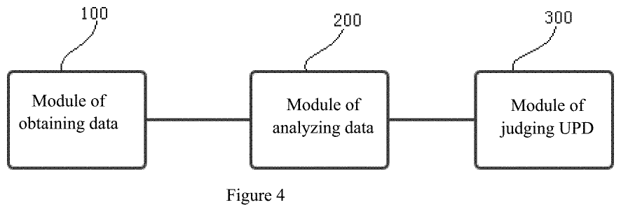

A device for screening uniparental disomy based upon NGS-trio, as shown in FIG. 4, comprises a module of obtaining data, a module of analyzing data, and a module of judging UPD.

The module of obtaining data was used to obtain NGS sequencing data of trio sample in a same group.

The module of analyzing data was used to analyze the above obtained data and classify mutation sites into loci in conformity with biparental inheritance, loci in conformity with uniparental inheritance only, and loci in inconformity with heredity law; the module of analyzing data was performed according to steps II to IV of the Example 1.

The module of judging UPD was used to perform UPD judgement on the above mutation sites according to a predetermined rule, to obtain a judgement result; the module of judging UPD was performed according to steps V to VIII of Example 1.

### Example 3

A UPD screening based upon NGS-trio was carried out in a clinical sample group (NP19E1936-NP19E1937-NP19F0086), by using the screening device of Example 2.

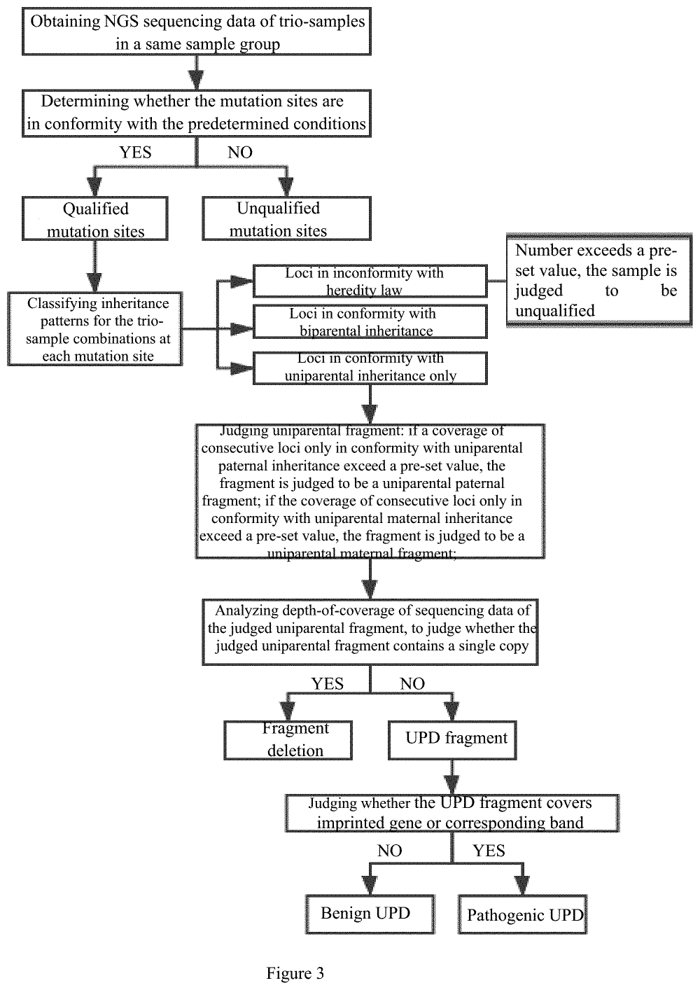

The result was shown in FIG. 3, which shown that there were almost only Norm (normal) loci in the above samples, while other types of loci were sporadic, which might be caused by sequencing errors or be due to new gene mutations in the genetic process. Therefore, the result indicated that the samples were all normal.

### Example 4

A UPD screening based upon NGS-trio was carried out, performed in 3 clinical sample groups as examples, by using the screening device of Example 2.

1. The trio sample group: NP21S0557-NP21S0558-NP21S0549.

The results were shown in FIGS. 4 and 5, which shown that there were loci in conformity with biparental inheritance, loci in conformity with uniparental inheritance only, and loci in inconformity with heredity law in the samples, and they were evenly distributed. And meanwhile, there were 11443 Type −1 loci and Type −2 loci, the number of which was more than 800. Therefore, the result indicated that the samples were unqualified, and both parents were non-biological, or the samples were error, and subsequent judgment had to be stopped.

2. The trio sample group: NP19E0911-NP19E0910-NP19E0912

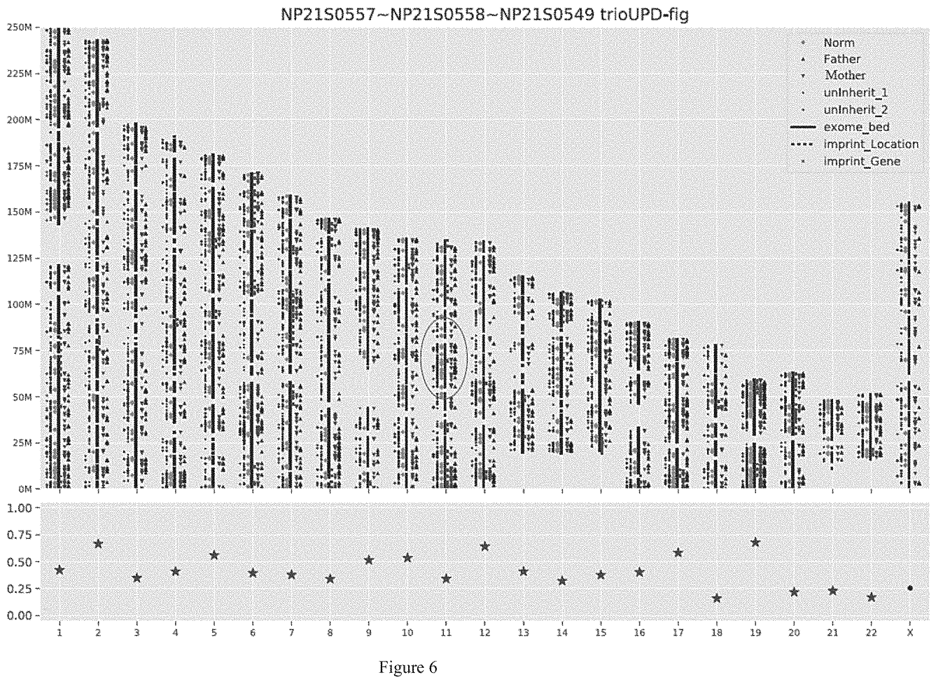

The results were shown in FIGS. 6 and 7, which shown that there were loci in conformity with biparental inheritance, loci in conformity with uniparental maternal inheritance only, and loci in inconformity with heredity law in the samples, and they were evenly distributed. Further, there was none loci in conformity with uniparental paternal inheritance (there were almost few Type 2F or Type 3F loci) in the samples, and meanwhile there were 5878 Type −1 loci and Type −2 loci, the number of which was more than 800. Therefore, the result indicated that the samples were unqualified, and the father was non-biological, or the samples were error, and subsequent judgment had to be stopped.

3. The trio sample group: NP20E957-NP20E956-NP20E958.

The results were shown in FIGS. 6 and 7, which shown that there were loci in conformity with biparental inheritance, loci in conformity with uniparental paternal inheritance only, and loci in inconformity with heredity law in the samples, and they were all evenly distributed. Further, there was none loci in conformity with uniparental maternal inheritance (there were almost few Type 2M or Type 3M loci) in the samples, and meanwhile there were 6044 Type −1 loci and Type −2 loci, the number of which was more than 800. Therefore, the result indicated the samples were unqualified, and the mother was non-biological, or the samples were error, and subsequent judgment had to be stopped.

After analyzing the above samples, the subsequent judgment had to be stopped, because these samples lacked of corresponding biological paternal sample or biological maternal sample, and did not meet the requirements of trio samples.

### Example 5

A UPD screening based on NGS-trio was carried out, performed in 3 clinical sample groups as examples, by using the screening device of Example 2.

1. The trio sample group: NP21F6166--NP21F6167-NP21F6168.

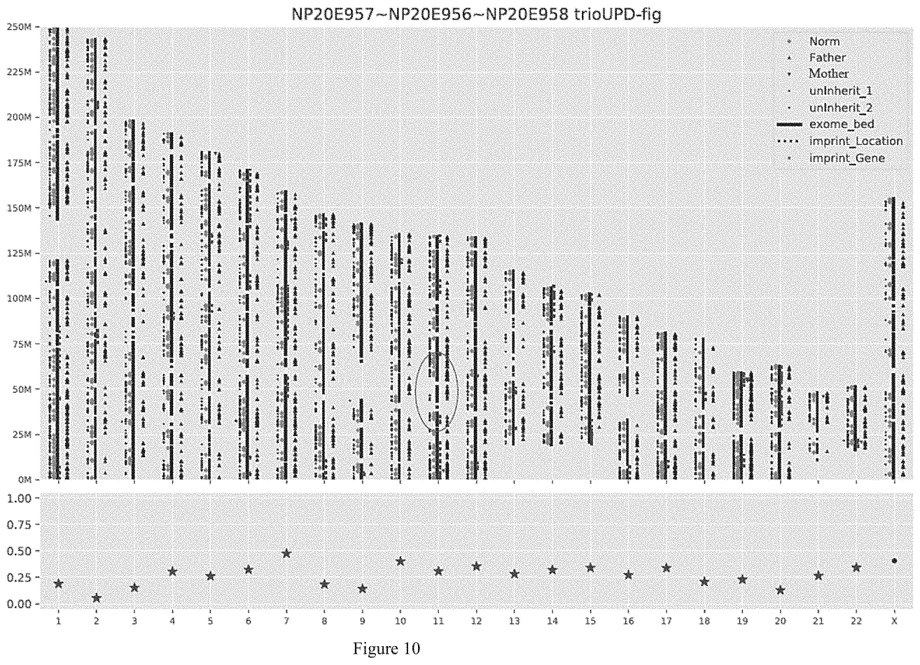

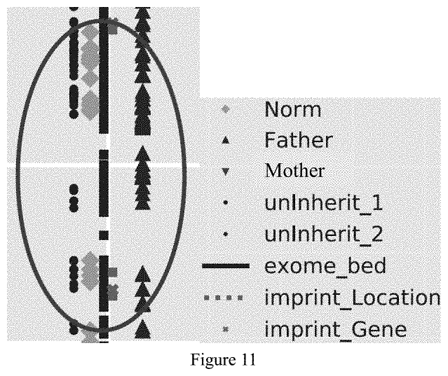

The results were shown in FIGS. 10 and 11, which shown that there were solely loci only in conformity with uniparental maternal inheritance in chr15 of the samples, while in other autosomes, almost all loci were ones in conformity with biparental inheritance and they were evenly distributed, and none of the loci was in conformity with uniparental paternal inheritance or in inconformity with heredity law (there were almost few Type 2F loci, Type 3F loci, Type −1 loci, or Type −2 loci). As there were 180 Type 2M loci or Type 3M loci in chr 15 with a coverage of 72 Mbp, and CNV result was normal, the samples were judged to be maternal UPD in chr15. Further, as the UPD fragment covered multiple imprinted genes, the samples were indicted to be at high risk for pathogenic UPD.

2. The trio sample group: NP19F0315--NP19F0313-NP19F0314

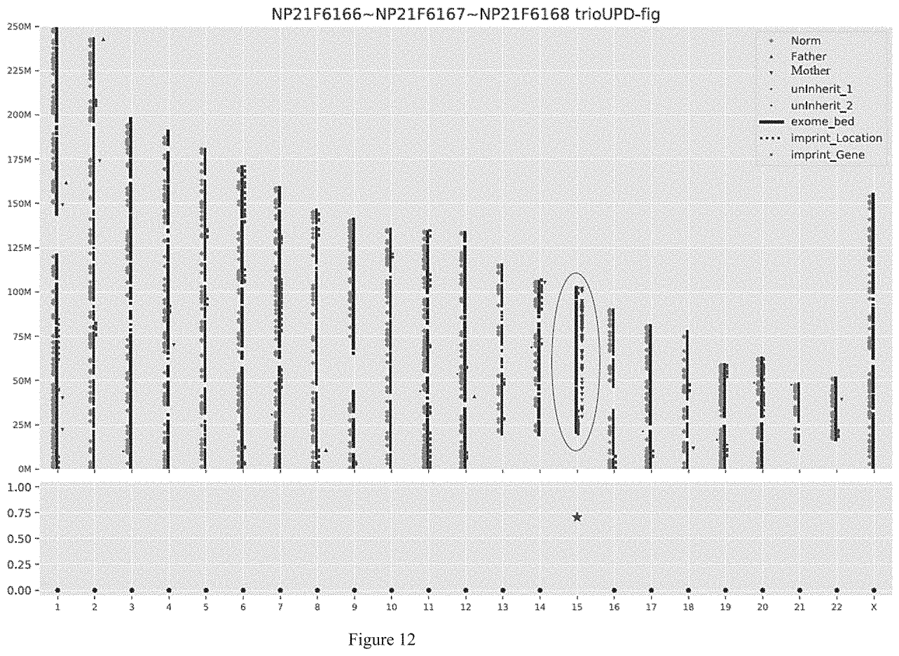

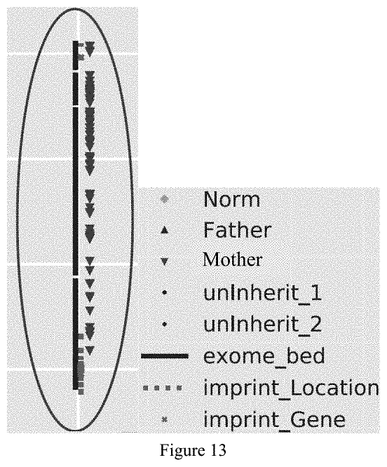

The results were shown in FIGS. 12 and 13, which shown that there were solely loci only in conformity with uniparental paternal inheritance in chr6 of the samples, while in other autosomes, almost all loci were ones in conformity with biparental inheritance and they were evenly distributed, and none of the loci was in conformity with uniparental maternal inheritance or in inconformity with heredity law (there were almost few Type 2M loci, Type 3M loci, Type −1 loci, or Type −2 loci). As there were 813 Type 2F loci or Type 3F loci in chr 6 with a coverage of 169 Mbp, and CNV result was normal, the samples were judged to be paternal UPD in chr6. Further, as the UPD fragment covered multiple imprinted genes, the samples were indicted to be at high risk for pathogenic UPD.

3. The trio sample group: NP21F3536--NP21F3567-NP21F3537.

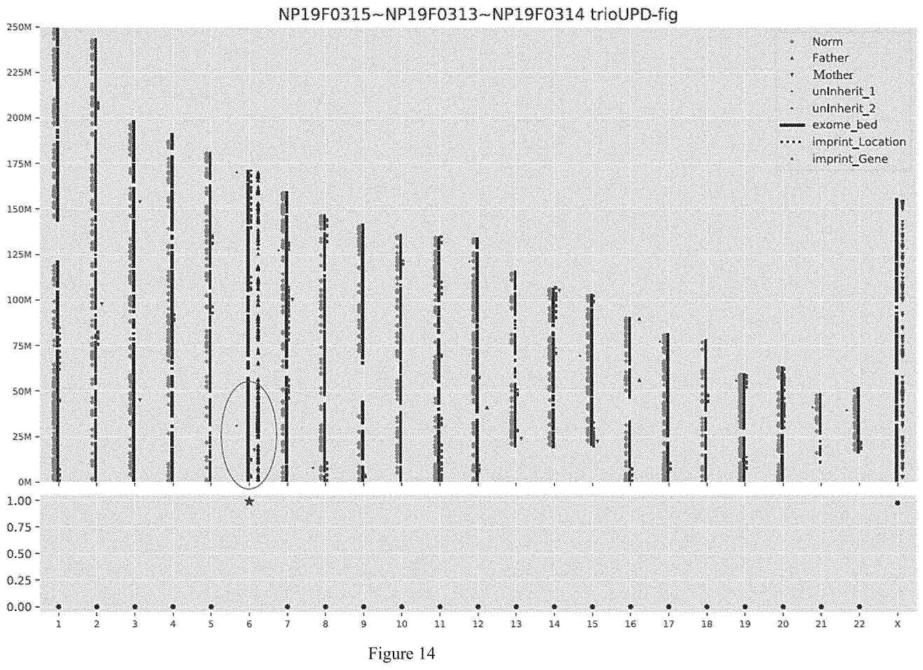

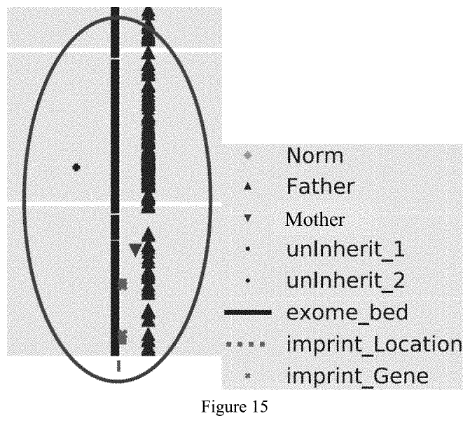

The results were shown in FIGS. 14 and 15, which shown that there were solely loci only in conformity with uniparental maternal inheritance in chr20 of the samples, while in other autosomes, almost all loci were ones in conformity with biparental inheritance and they were evenly distributed, and none of the loci was in conformity with uniparental paternal inheritance or in inconformity with heredity law (there were almost few Type 2F loci, Type 3F loci, Type −1 loci, or Type −2 loci). As there were 197 Type 2M loci or Type 3M loci in chr 20 with a coverage of 63 Mbp, and CNV result was normal, the samples were judged to be maternal UPD in chr20. Further, as the UPD fragment covered multiple imprinted genes, the samples were indicted to be at high risk for pathogenic UPD.

After analysis, the above samples were all at risk for pathogenic UPD.

### Example 6

A UPD screening based on NGS-trio was carried out, performed in 2 clinical sample groups as examples, by using the screening device of Example 2.

1. The trio sample group: NP19E1380--NP19E1381-NP19E1382.

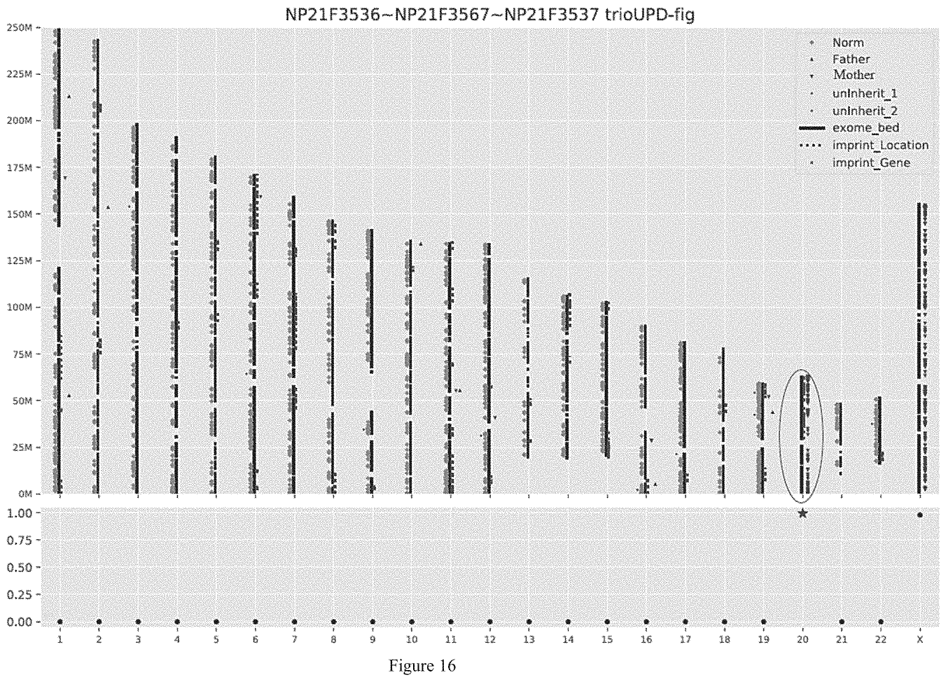

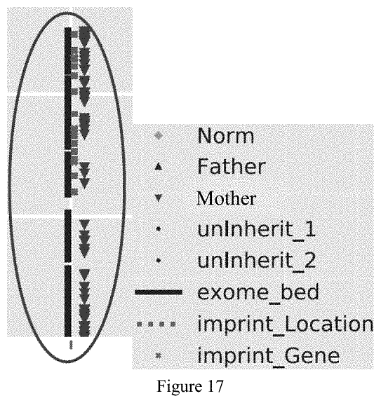

The results were shown in FIGS. 16 and 17, which shown that there were solely loci only in conformity with uniparental paternal inheritance in a portion of chr15 of the samples, while in the remaining portions of chr 15 and other autosomes, almost all loci were those in conformity with biparental inheritance and they were evenly distributed, and none of the loci was in conformity with uniparental maternal inheritance or in inconformity with heredity law (there were almost few Type 2M loci, Type 3M loci, Type −1 loci, or Type −2 loci). As there were 16 consecutive Type 2F loci or Type 3F loci in chr 15 with a coverage of 4 Mbp, and CNV result indicated that there was a 4 Mbp of heterozygous deletion at the same loci of chr 15, the samples were judged to be partial deletion of maternal chr15, that is, there was only one copy of paternal inheritance for partial fragments of chr 15 (which may lead to the same clinical effect as paternal UPD). As the fragments covered multiple imprinted genes, the samples were indicted to be at high risk for pathogenic maternal heterozygous deletion.

2. The trio sample group: NP19E0056--NP9E0057-NP9E0055

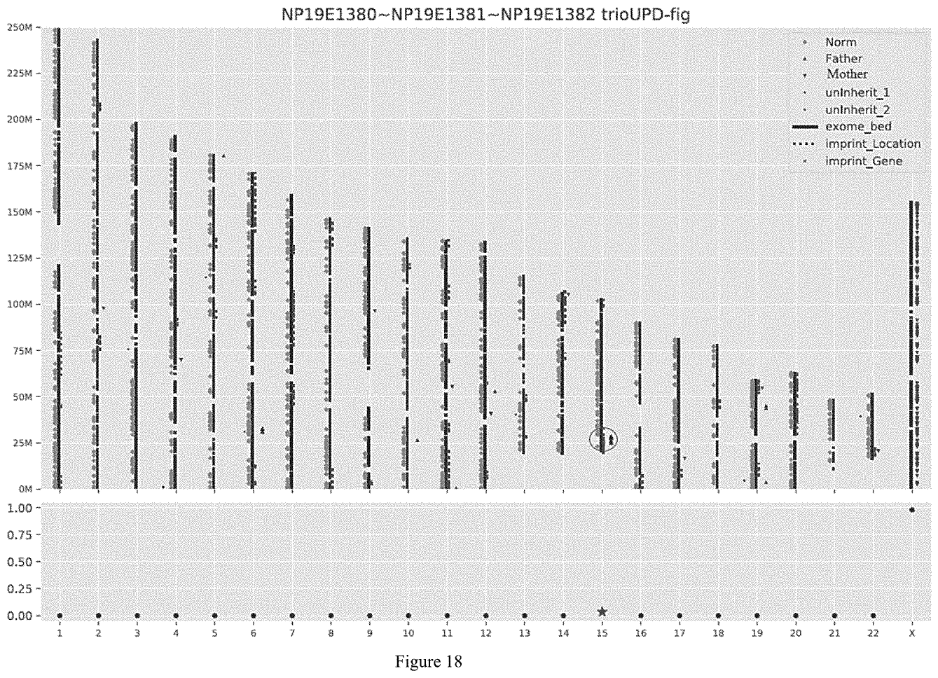

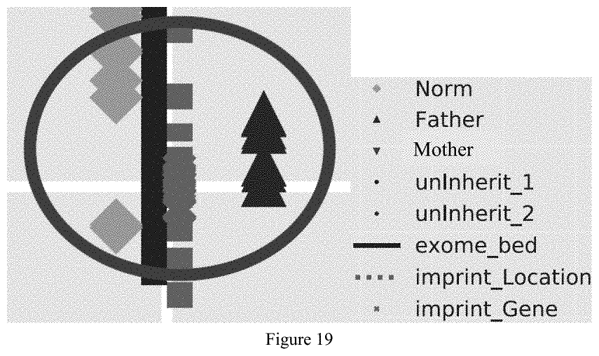

The results were shown in FIGS. 18 and 19, which shown that there were solely loci only in conformity with uniparental maternal inheritance in a portion of chr8 of the samples (though there was one uniparental paternal locus, which may be caused by sequencing error or other reasons, the analysis could not be affected), while in the remaining portions of chr 8 and other autosomes, almost all loci were those in conformity with biparental inheritance and they were evenly distributed, and none of the loci was in conformity with uniparental paternal inheritance or in inconformity with heredity law (there were almost few Type 2M loci, Type 3M loci, Type −1 loci, or Type −2 loci). As there were 69 consecutive Type 2M loci or Type 3M loci in chr 8 with a coverage of 11 Mbp, and CNV result indicated that there was a 11 Mbp of heterozygous deletion at the same sites of chr 8, the samples were judged to be partial deletion of paternal chr8, that is, there was only one copy of maternal inheritance for partial fragments of chr 8 (which may lead to the same clinical effect as maternal UPD). As the fragments covered multiple imprinted genes, the samples were indicted to be at high risk for pathogenic paternal heterozygous deletion.

After analysis, the above samples were all at high-risk for pathogenic heterozygous deletion, and they had the same clinical effects as that of the UPD which had an opposite origin of deletion.

### Example 7

The screening device of Example 2 was used to screen UPD from 792 samples in whole exome trio sequencing, and the results were shown as follows.

TABLE 1
Results of screening for UPD from 792 sample groups in whole exome trio
sequencing

| | | |
|---|---|---|
| Total groups for Screening | 792 | groups |
| Non-biological relationship | 5 | groups |
| Detected uniparental origin | 46 (14+32) | groups |
| Heterozygous deletion | 32 | groups |
| (being consistent to CNV result ) | | |
| UPD | 14 | groups |
| PWS-AS region detected by the present disclosure/ | 7/7 | groups |
| validated by Methylated detection method | | |

Note:
the above-described ″detected uniparental origin″ refers to detected UPD (14 groups) or heterozygous deletion (32 groups); the above-described ″PWS-AS″ refers to a pathogenic condition caused by chr 15-UPD, wherein maternal UPD may cause PWS, paternal UPD may cause AS, chr 15-UPD is a common pathogenic condition, and a methylation detection method is available for detecting the chr 15-UPD on market nowadays. For the chr15-UPD, maternal UPD may cause PWS, and paternal UPD may cause AS. The 7 chr 15-PD samples screened out in the Example 6 were all verified by the methylation detection method, and the results are consistent with those of the present disclosure, indicating that the method of the present disclosure has a high accuracy of detection results

The technical features of the above embodiments can be combined arbitrarily. For the sake of brief description, not all possible combinations of the technical features in the above embodiments are described. However, the combination of these technical features should be considered within the scope of the present specification as long as they are not contradictory.

The above embodiments only express several implementation modes of the present invention, which are described concretely and detailly and cannot be understood as a limitation of the invention patent scope. It should be pointed out that, for ordinary one skilled in the art, several modifications and improvements can also be made, which are within the protection scope of the invention, without departing from the contents of the present disclosure. Therefore, the protection scope of the invention patent shall be subject to the appended claims.

1. A method for detecting a uniparental disomy based upon NGS-trio, comprising steps as follows:
obtaining data: obtaining NGS sequencing data of trio-samples in a same sample group;
screening for mutation sites: selecting mutation sites which are in conformity with pre-determined conditions in each trio-sample, respectively and defining such mutation sites as qualified mutation sites of corresponding trio-samples, and defining un-selected mutation sites as unqualified mutation sites;
merging mutation site data: merging the unqualified mutation sites from all the trio-samples in the same sample group, obtaining and gathering a chromosome coordinate of each unqualified mutation site, removing mutation sites which have identical chromosome coordinates to chromosome coordinates of the unqualified mutation sites from the qualified mutation sites in each trio-sample; and based on the remaining qualified mutation sites of the samples in the sample group, defining genotypes of the non-mutation sites as genotypes of homozygous sites, which are consistent with genotypes of a reference sequence;
classifying inheritance pattern: classifying inheritance patterns for trio-sample combinations at each mutation site, wherein the mutation sites is classified into loci in conformity with biparental inheritance, loci in conformity with uniparental inheritance only, and loci in inconformity with heredity law;
judging genetic relationship: if the number of the loci in inconformity with heredity law is smaller than a pre-set value, a follow-up analysis is performed; if the number of the loci in inconformity with heredity law is larger than the pre-set value, the sample is judged to be unqualified;
judging uniparental fragment: if a coverage of consecutive loci which are only in conformity with uniparental paternal inheritance exceed a pre-set value, the fragment is judged to be a uniparental paternal fragment; if the coverage of consecutive loci which are only in conformity with uniparental maternal inheritance exceed a pre-set value, the fragment is judged to be a uniparental maternal fragment;
judging UPD: analyzing depth-of-coverage of sequencing data of the judged uniparental fragment, wherein if the judged uniparental fragment contains a single copy, it is judged that fragment deletion occurs in the uniparental fragment, otherwise, the uniparental fragment is judged as a UPD fragment; and
screening pathogenic UPD: determining whether the UPD fragment covers imprinted gene or corresponding band, wherein if the UPD fragment does not cover the imprinted gene or corresponding band, the UPD fragment is judged to be benign UPD, otherwise, the UPD fragment is judged to be pathogenic UPD.

2. The method for detecting a uniparental disomy based upon NGS-trio according to claim 1, wherein in the step of screening for mutation sites, the mutation sites are obtained as follows:
1) screening for high-quality mutation sites from NGS sequencing data;
2) removing Y chromosome mutation sites from the above mutation sites;
3) screening for single nucleotide substitutions from the mutation sites obtained in the step of removing Y chromosome mutation sites;
4) excluding uncertain false positive single nucleotide substitutions according to Hardy-Weinberg equilibrium;
5) removing heterozygous sites which have a mutation frequency of more than 70% and homozygous sites which have a mutation frequency of less than 85%;
6) classifying a genotype of the mutation for each site, and removing sites with more than 2 genotypes; and
7) determining remaining sites, which meet the predetermined conditions.

3. The method for detecting a uniparental disomy based upon NGS-trio according to claim 1, wherein in the step of screening for mutation sites, the high-quality mutation sites are mutation sites passed through a quality control of GATK-VQSR, and having a total coverage range of more than 20× and a mutation frequency of greater than 25%.

4. The method for detecting a uniparental disomy based upon NGS-trio according to claim 1, wherein in the step of obtaining data, the trio samples in the same group comprise a paternal sample, a maternal sample and a proband sample; and
in the step of merging mutation site data, the mutation sites, which have identical coordinate, are arranged in an order of proband, father, mother.

5. The method for detecting a uniparental disomy based upon NGS-trio according to claim 4, wherein in the step of classifying inheritance pattern, the loci in conformity with biparental inheritance are classified into:
Type 1: loci only in conformity with biparental inheritance; and
Type 0: loci in conformity with both biparental inheritance and uniparental inheritance;
wherein the loci only in conformity with uniparental inheritance are classified into:
Type 3F: loci only produced by paternal monosomy rescue;
Type 2F: loci produced by either paternal monosomy rescue or paternal trisomy rescue;
Type 3M: loci only produced by maternal monosomy rescue; and
Type 2M: loci produced by either maternal monosomy rescue or maternal trisomy rescue;
wherein the loci in inconformity with heredity law are classified into:
Type −1: loci from either of parent in inconformity with heredity law; and
Type −2: loci from both parents in inconformity with heredity law.

6. The method for detecting a uniparental disomy based upon NGS-trio according to claim 5, wherein in the step of judging uniparental fragment, if there are more than 8 Type 2F loci or Type 3F loci with a coverage of more than 1 Mbp, the fragment is judged to be a uniparental paternal fragment; if there are more than 8 Type 2M loci or Type 3M loci with a coverage of more than 1 Mbp, the fragment is judged to be a uniparental maternal fragment.

7. The method for detecting a uniparental disomy based upon NGS-trio according to claim 1, wherein in the step of judging UPD, the data of the judged uniparental fragment is compared with analysis results of copy number of whole exome sequencing, and if the analysis result of copy number indicates that the judged uniparental fragment contains a single copy, it is judged that fragment deletion occurs in the uniparental fragment; if not, the uniparental fragment is judged to be a UPD fragment.

8. A method of developing or manufacturing a device for screening UPD, comprising applying the method of detecting a uniparental disomy based upon NGS-trio according to claim 1.

9. A device for screening a uniparental disomy based upon NGS-trio, comprising a module of obtaining data, a module of analyzing data, and a module of judging UPD; wherein
the module of obtaining data is used to obtain NGS sequencing data of trio samples in a same group;
the module of analyzing data is used to analyze the above obtained data and classify mutation sites into loci in conformity with biparental inheritance, loci in conformity with uniparental inheritance only, and loci in inconformity with heredity law; and
the module of judging UPD is used to perform UPD judgement on the above mutation sites according to a predetermined rule, to obtain a judgement result;
the module of analyzing data is conducted in following steps:
screening for mutation sites: selecting mutation sites which are in conformity with pre-determined conditions in each trio-sample, respectively and defining such mutation sites as qualified mutation sites of the corresponding trio-samples, and defining un-selected mutation sites as unqualified mutation sites;
merging mutation site data: merging all the unqualified mutation sites from the trio-samples in the same sample group, obtaining and gathering chromosome coordinates of each unqualified mutation site, removing mutation sites which have identical chromosome coordinates to chromosome coordinates of the unqualified mutation sites from the qualified mutation sites in each trio-sample; and based on the remaining qualified mutation sites in this group of the samples, defining genotypes of the non-mutation sites as genotypes of homozygous sites, which are consistent with genotypes of a reference sequence; and
classifying inheritance pattern: classifying inheritance patterns for trio-sample combinations at each mutation site, wherein the mutation sites is classified into loci in conformity with biparental inheritance, loci in conformity with uniparental inheritance only, and loci in inconformity with heredity law;
wherein the module of judging UPD is conducted in following steps:
judging genetic relationship: if the number of the loci in inconformity with heredity law is smaller than a pre-set value, a follow-up analysis is performed; if the number of the loci in inconformity with heredity law is larger than the pre-set value, the sample is judged to be unqualified;
judging uniparental fragment: if a coverage of consecutive loci which are only in conformity with uniparental paternal inheritance exceed a pre-set value, the fragment is judged to be a paternal fragment; if the coverage of consecutive loci which are only in conformity with uniparental maternal inheritance exceed a pre-set value, the fragment is judged to be a maternal fragment;
judging UPD: analyzing depth-of-coverage of the sequencing data of the judged uniparental fragment, wherein if the judged uniparental fragment contains a single copy, it is judged that fragment deletion occurs in the uniparental fragment,; otherwise, the uniparental fragment is judged as a UPD fragment; and
screening pathogenic UPD: determining whether the UPD fragment covers imprinted gene or corresponding band, wherein if the UPD fragment does not cover the imprinted gene or corresponding band, the UPD fragment is judged to be benign UPD, otherwise, the UPD fragment is judged to be pathogenic UPD.

10. The device for screening a uniparental disomy based upon NGS-trio according to claim 9, wherein
in the step of screening for mutation sites, the mutation sites are obtained as follows:
1) screening for high-quality mutation sites from NGS sequencing data;
2) removing Y chromosome mutation sites from the above mutation sites;
3) screening for single nucleotide substitutions from the mutation sites obtained in the step of removing Y chromosome mutation sites;
4) excluding uncertain false positive single nucleotide substitutions according to Hardy-Weinberg;
5) removing heterozygous sites which have a mutation frequency of more than 70% and homozygous sites which have a mutation frequency of less than 85%;
6) classifying a genotype of the mutation at each site, and removing sites with more than 2 genotypes; and
7) determining remaining sites, which meet a predetermined condition.

11. The device for screening a uniparental disomy based upon NGS-trio according to claim 9, wherein in the step of screening for mutation sites, the high-quality mutation sites are mutation sites passed through a quality control of GATK-VQSR, and having a total coverage range of more than 20× and a mutation frequency of greater than 25%.

12. The device for screening a uniparental disomy based upon NGS-trio according to claim 9, wherein in the module of obtaining data, the trio samples in the same group comprise a paternal sample, a maternal sample and a proband sample.
in the step of merging mutation site data, the mutation sites, which have identical coordinate, are arranged in an order of proband, father, mother.

13. The device for screening a uniparental disomy based upon NGS-trio according to claim 12, wherein in the step of classifying inheritance pattern, the loci in conformity with biparental inheritance are classified into:
Type 1: loci only in conformity with biparental inheritance; and
Type 0: loci in conformity with both biparental inheritance and uniparental inheritance;
the loci only in conformity with uniparental inheritance are classified into:
Type 3F: loci only produced by paternal monosomy rescue;
Type 2F: loci produced by either paternal monosomy rescue or paternal trisomy rescue;
Type 3M: loci only produced by maternal monosomy rescue; and
Type 2M: loci produced by either maternal monosomy rescue or maternal trisomy rescue;
the loci in inconformity with heredity law are classified into:
Type −1: loci from either of parent in inconformity with heredity law; and
Type −2: loci from both parents in inconformity with heredity law.

14. The device for screening a uniparental disomy based upon NGS-trio according to claim 13, wherein in the step of judging uniparental fragment, if there are more than 8 Type 2F loci or Type 3F loci with a coverage of more than 1 Mbp, the fragment is judged to be uniparental paternal fragment; if there are more than 8 Type 2M loci or Type 3M loci with a coverage of more than 1 Mbp, the fragment is judged to be uniparental maternal fragment.

15. The device for screening a uniparental disomy based upon NGS-trio according to claim 9, wherein in the step of judging UPD, the data of the judged uniparental fragment is compared with the analysis results of copy number of whole exome sequencing, and if the analysis result of copy number indicates that the judged uniparental fragment contains a single copy, it is judged that fragment deletion occurs in the judged uniparental fragment; if not, the uniparental fragment is judged to be a UPD fragment.

16. A storage medium, comprising a stored program which achieves functions of the modules according to claim 9.

17. A processor, for running a program that realizes the functions of the modules according to claim 9.
